(19) United States
(12) Patent Application Publication
(10) Pub. No.: US 2022/0047589 A1
(43) Pub. Date: Feb. 17, 2022

(54) PDE9 INHIBITORS FOR TREATING THALASSEMIA

(71) Applicant: Imara Inc., Boston, MA (US)

(72) Inventors: Rahul Dilip BALLAL, Boston, MA (US); Thiago Trovati MACIEL, Paris (FR)

(21) Appl. No.: 17/514,337

(22) Filed: Oct. 29, 2021

Related U.S. Application Data
(63) Continuation of application No. PCT/US2020/031659, filed on May 6, 2020.
(60) Provisional application No. 62/844,571, filed on May 7, 2019.

(51) Int. Cl. A61K 31/4985; A61K 45/06; A61P 7/06

(52) U.S. Cl. CPC A61K 31/4985; A61P 7/06; A61K 45/06

(57) ABSTRACT

The present disclosure relates to PDE9 inhibitors, compositions comprising the PDE9 inhibitors, and methods of using the PDE9 inhibitors and compositions for treatment of thalassemia.

### CROSS-REFERENCE TO RELATED APPLICATIONS

This application is a continuation of International Application No. PCT/US2020/031659, filed on May 6, 2020, which claims the benefit of U.S. Provisional Patent Application No. 62/844,571, filed on May 7, 2019, and is incorporated herein by reference in its entirety.

### BACKGROUND OF THE INVENTION

Thalassemia disorders (referred to as thalassemia in the present disclosure) are inherited blood disorders characterized by less hemoglobin and reduced red blood cells in the body as compared to normal. The low hemoglobin and red blood cell levels of thalassemia may cause symptoms such as anemia, drowsiness, fatigue, chest pain, and shortness of breath. Alpha thalassemia and beta thalassemia are two main types of thalassemia; they both have major and minor forms. Beta thalassemia major is also called Cooley anemia or Cooley thalassemia. Beta thalassemia also includes beta-plus thalassemia and beta-zero thalassemia. Beta thalassemia patients have genetic defects resulting in the synthesis of little or no hemoglobin beta chains. Symptoms of beta thalassemia include anemia, a lack of oxygen in many parts of the body, pulmonary hypertension, thrombotic events, infection, endocrine dysfunction and leg ulcers

Treatments of thalassemia such as blood transfusions and iron chelation can be helpful, but this disease cannot be cured. Repeated transfusions may also cause iron overload and many side effects.

### SUMMARY OF THE DISCLOSURE

The present disclosure provides methods of making and using Compound 1 and/or pharmaceutical compositions comprising Compound 1 or a pharmaceutically acceptable salt, solvate, or polymorph thereof, to treat thalassemia.

An aspect of the present disclosure comprises a method of treating thalassemia comprising administering 6-[4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or polymorph thereof to a subject in need thereof. In some embodiments, the administration results in an increase of the hemoglobin level (Hb) of the subject. In some embodiments, the subject's hemoglobin level (Hb) is increased in the range of about 0.5 to about 3.0 g/dL of total Hb. In some embodiments, the subject's hemoglobin level (Hb) is increased by about 0.5, about 1.0, about 1.5, about 2.0, about 2.5, or about 3.0 g/dL of total Hb. In some embodiments, the administration results in an increase of the red blood cell (RBC) level of the subject. In some embodiments, the RBC level is increased by at least 5%, 10%, 25%, or 50% over baseline RBC level prior to the administration of Compound 1. In some embodiments, the administration results in a decrease of the immature red blood cell level of the subject. In some embodiments, the immature red blood cell level of the subject is decreased by at least 5%, 10%, 25%, or 50% over baseline level prior to the administration of Compound 1. In some embodiments, the administration results in an increase of the mature red blood cell level of the subject. In some embodiments, the mature red blood cell level of the subject is increased by at least 5%, 10%, 25%, or 50% over baseline level prior to the administration of Compound 1. In some embodiments, the administration results in an increase in the red blood cell (RBC) maturation ratio of the subject prior to administration. In some embodiments, the red blood cell (RBC) maturation ratio is increased by at least 5%, 10%, 25%, or 50%. In some embodiments, the method increases hemoglobin (Hb) levels, red blood cell (RBC) levels, or levels of mature red blood cells greater than traditional blood transfusion. In some embodiments, Compound 1, or a pharmaceutically acceptable salt, solvate, or polymorph thereof, is administered orally. In some embodiments, Compound 1, or a pharmaceutically acceptable salt, solvate, or polymorph thereof, is administered daily. In some embodiments, Compound 1, or a pharmaceutically acceptable salt, solvate, or polymorph thereof, is administered with food or without food. In some embodiments, Compound 1, or a pharmaceutically acceptable salt, solvate, or polymorph thereof, is administered for between 1 to 7 days. In some embodiments, Compound 1, or a pharmaceutically acceptable salt, solvate, or polymorph thereof, is administered for at least 7 days. The method of any one of claims 1-20, wherein Compound 1, or a pharmaceutically acceptable salt, solvate, or polymorph thereof, is administered with an additional therapy. In some embodiments, the additional therapy is a gene therapy a bone and/or marrow stem cell transplant a blood transfusion, or an iron chelation therapy. In some embodiments, the thalassemia is beta thalassemia. In some embodiments, the thalassemia is beta-plus thalassemia or beta-zero thalassemia. In some embodiments, the thalassemia is major beta thalassemia or minor beta thalassemia. In some embodiments, the thalassemia is alpha thalassemia. In some embodiments, the thalassemia is major alpha thalassemia or minor alpha thalassemia.

Another aspect of the disclosure described herein comprises a method of treating thalassemia comprising administering Compound 1 to a subject in need thereof. In some embodiments, the administration results in an increase of the hemoglobin level of the subject. In some embodiments, the subject's hemoglobin level (Hb) is increased in the range of about 0.5 to about 3.0 g/dL of total Hb. In some embodiments, the administration results in an increase of the red blood cell level of the subject. In some embodiments, the administration results in a decrease of the immature red blood cell level of the subject. In some embodiments, the administration results in an increase of the mature red blood cell level of the subject. In some embodiments, Compound 1 is administered daily. In some embodiments, the thalassemia is beta thalassemia. In some embodiments, the thalassemia is beta-plus thalassemia or beta-zero thalassemia. In some embodiments, the thalassemia is major beta thalassemia or minor beta thalassemia. In some embodiments, the thalassemia is alpha thalassemia. In some embodiments, the thalassemia is major alpha thalassemia or minor alpha thalassemia. In some embodiments, the method increases hemoglobin levels, red blood cell levels, or levels of mature red blood cells greater than traditional blood transfusion.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A and FIG. 1B show Compound 1 treatment increases the total Hb amount (FIG. 1A) and the RBC amount (FIG. 1B).

FIG. 2A shows Compound 1 treatment reduces immature RBC levels. FIG. 2B shows Compound 1 treatment increases mature RBC levels. FIG. 2C shows Compound 1 treatment increases RBC maturation ratio.

### DETAILED DESCRIPTION OF THE INVENTION

Phosphodiesterases (PDEs) are a family of enzymes degrading cyclic nucleotides and thereby regulating the cellular levels of second messengers throughout the entire body. PDEs represent attractive drug targets, as proven by a number of compounds that have been introduced to clinical testing and the market, respectively. PDEs are encoded by 21 genes that are functionally separated into 11 families differing with respect to kinetic properties, substrate selectivity, expression, localization pattern, activation, regulation factors and inhibitor sensitivity. The function of PDEs is the degradation of the cyclic nucleotide monophosphates cyclic Adenosine Monophosphate (cAMP) and/or Guanosine Monophosphate (cGMP), which are important intracellular mediators involved in numerous vital processes including the control of neurotransmission and smooth muscle contraction and relaxation.

PDE9 is cGMP specific (Km cAMP is >1000× for cGMP) and is hypothesized to be a key player in regulating cGMP levels as it has the lowest Km among the PDEs for this nucleotide. PDE9 is expressed throughout the brain at low levels with the potential for regulating basal cGMP.

In the periphery, PDE9 expression is highest in prostate, intestine, kidney and hematopoietic cells, enabling therapeutic potential in various non-CNS indications.

In the present disclosure, a PDE9 inhibitor (for example, Compound 1) is used for treatment for thalassemia.

### I. Compounds of the Disclosure

In the context of the present disclosure a compound is considered to be a PDE9 inhibitor if the amount required to reach the 50% inhibition level of any of the three PDE9 isoforms is 10 micromolar or less, preferably less than 9 micromolar, such as 8 micromolar or less, such as 7 micromolar or less, such as 6 micromolar or less, such as 5 micromolar or less, such as 4 micromolar or less, such as 3 micromolar or less, more preferably 2 micromolar or less, such as 1 micromolar or less, in particular 500 nM or less. In preferred embodiments the required amount of PDE9 inhibitor required to reach the IC₅₀ level of PDE9 is 400 nM or less, such as 300 nM or less, 200 nM or less, 100 nM or less, or even 80 nM or less, such as 50 nM or less, for example 25 nM or less.

Throughout this application the notations IC₅₀ and IC50 are used interchangeably.

In some embodiments, the PDE9 inhibitor of the present disclosure has low or no blood brain barrier penetration. For example, the ratio of the concentration of a PDE9 inhibitor of the present disclosure in the brain to the concentration of it in the plasma (brain/plasma ratio) may be less than about 0.50, about 0.40, about 0.30, about 0.20, about 0.10, about 0.05, about 0.04, about 0.03, about 0.02, or about 0.01. The brain/plasma ratio may be measured 30 min or 120 min after administration of the PDE9 inhibitor.

In some embodiments, the PDE9 inhibitor may be any imidazo pyrazinone PDE9 inhibitor disclosed in WO 2013/053690 and/or any imidazo triazinone PDE9 inhibitor disclosed in WO 2013/110768, the contents of each of which are incorporated herein by reference in their entirety.

In some embodiments, the PDE9 inhibitor is Compound 1 or a pharmaceutically acceptable salt, solvate or polymorph thereof. A racemate form of Compound 1 and an anhydrous form of Compound 1 have been described in WO 2013/053690 and WO 2017/005786. Crystalline forms have been described in WO 2019/226944. Compound 1 has the following structure:

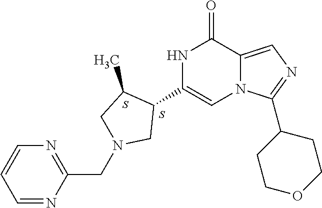

6-[(3S,4S)-4-methyl-1-(pyrimidin-2-ylmethyl)pyrrolidin-3-yl]-3-tetrahydropyran-4-yl-7H-imidazo[1,5-a]pyrazin-8-one; Formula C₂₁H₂₆N₆O₂; calculated molecular weight about 394 g/mol.

### II. Pharmaceutical Composition

The present disclosure further provides a pharmaceutical composition comprising a therapeutically effective amount of any of the PDE9 inhibitors and a pharmaceutically acceptable carrier or diluent. In some embodiments, the present disclosure provides a pharmaceutical composition comprising a therapeutically effective amount of Compound 1 or a pharmaceutically acceptable salt, solvate, or polymorph thereof, and a pharmaceutically acceptable carrier or diluent or excipient.

### Pharmaceutically Acceptable Salts

The present disclosure also comprises salts of the PDE9 inhibitors, typically, pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutically acceptable inorganic or organic acid addition salts include the pharmaceutically acceptable salts listed in Berge, S. M. et al., J. Pharm. Sci. 1977, 66, 2, the contents of which are hereby incorporated by reference.

Furthermore, the compounds of this disclosure may exist in unsolvated as well as in solvated forms with pharmaceutically acceptable solvents such as water, ethanol and the like. In general, the solvated forms are considered equivalent to the unsolvated forms for the purposes of this disclosure.

In some embodiments, the pharmaceutical composition comprises Compound 1 as the solvated, unsolvated, or crystalline/polymorph form. In some embodiments, Compound 1 is present as the unsolvated form. In some embodiments, Compound 1 is present as the solvated form. In some embodiments, Compound 1 is present as the crystalline form. In some embodiments, Compound 1 is present as the monohydrate crystalline form.

### Formulations

The compounds of the disclosure may be administered alone or in combination with pharmaceutically acceptable carriers, diluents or excipients, in either single or multiple doses. The pharmaceutical compositions according to the disclosure may be formulated with pharmaceutically acceptable carriers or diluents as well as any other known adjuvants and excipients in accordance with conventional techniques such as those disclosed in Remington: The Science and Practice of Pharmacy, 22nd Edition, Gennaro, Ed., Mack Publishing Co., Easton, Pa., 2013.

The pharmaceutical compositions may be specifically formulated for administration by any suitable route such as oral, rectal, nasal, pulmonary, topical (including buccal and sublingual), transdermal, intracisternal, intraperitoneal, vaginal and parenteral (including subcutaneous, intramuscular, intrathecal, intravenous and intradermal) routes. It will be appreciated that the route will depend on the general health and age of the subject to be treated, the nature of the condition to be treated and the active ingredient.

Pharmaceutical compositions for oral administration include solid dosage forms such as capsules, tablets, dragees, pills, lozenges, powders and granules. Where appropriate, the compositions may be prepared with coatings such as enteric coatings or they may be formulated so as to provide controlled release of the active ingredient such as sustained or prolonged release according to methods well known in the art. Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Other suitable administration forms include, but are not limited to, suppositories, sprays, ointments, creams, gels, inhalants, dermal patches and implants.

For parenteral routes such as intravenous, intrathecal, intramuscular and similar administration, typical doses are on the order of half the dose employed for oral administration.

The present disclosure also provides a process for making a pharmaceutical composition comprising admixing a therapeutically effective amount of a compound of the present disclosure and at least one pharmaceutically acceptable carrier or diluent.

The compounds of this disclosure are generally utilized as the free substance or as a pharmaceutically acceptable salt thereof. Such salts are prepared in a conventional manner by treating a solution or suspension of a compound of the present disclosure with a molar equivalent of a pharmaceutically acceptable acid. Representative examples of suitable organic and inorganic acids are described above.

For parenteral administration, solutions of the compounds of the present disclosure in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The compounds of the present disclosure may be readily incorporated into known sterile aqueous media using standard techniques known to those skilled in the art.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of the present disclosure and a pharmaceutically acceptable carrier are then readily administered in a variety of dosage forms suitable for the disclosed routes of administration. The formulations may conveniently be presented in unit dosage form by methods known in the art of pharmacy.

Formulations of the present disclosure suitable for oral administration may be presented as discrete units such as capsules or tablets, each containing a predetermined amount of the active ingredient, and optionally a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatin capsule in powder or pellet form or it may be in the form of a troche or lozenge. The amount of solid carrier will vary widely but will range from about 25 mg to about 1 g per dosage unit. If a liquid carrier is used, the preparation may be in the form of a syrup, emulsion, soft gelatin capsule or sterile injectable liquid such as an aqueous or non-aqueous liquid suspension or solution.

The pharmaceutical compositions of the disclosure may be prepared by conventional methods in the art. For example, tablets may be prepared by mixing the active ingredient with ordinary adjuvants and/or diluents and subsequently compressing the mixture in a conventional tabletting machine prepare tablets. Examples of adjuvants or diluents comprise: corn starch, potato starch, talcum, magnesium stearate, gelatin, lactose, gums, and the like. Any other adjuvants or additives usually used for such purposes such as colorings, flavorings, preservatives etc. may be used provided that they are compatible with the active ingredients.

The pharmaceutical compositions may comprise at least 10%, 20%, 30%, 40%, 50%, 60%, 70%, 80%, or 90% by weight of PDE9 inhibitor Compound 1, or a pharmaceutically acceptable salt, solvate, or polymorph thereof. In some embodiments, the pharmaceutical composition may comprise at least 90%, 91%, 92%, 93%, 94%, 95%, 96%, 97%, 98%, or 99% by weight of Compound 1, or a pharmaceutically acceptable salt, solvate, or polymorph thereof.

In some embodiments, Compound 1 or a pharmaceutically acceptable salt, solvate, or polymorph thereof is formulated as a composition for oral administration. For example, it may be in a solid tablet form. The composition for oral administration comprises at least a filler and/or a processing aid. The processing aid may be a glidant or a lubricant. The composition for oral administration may also comprise a coating. In some embodiments, the composition for oral administration comprises microcrystalline cellulose and/or pregelatinized starch as fillers. In some embodiments, the composition for oral administration comprises colloidal silicon dioxide and/or magnesium stearate as processing aids. In some embodiments, the composition for oral administration comprises Opadry® II white film coating. Opadry® II is a high productivity, water soluble, pH independent complete dry powder film coating system containing polymer, plasticizer and pigment which allows for immediate disintegration for fast, active release. In some embodiments, the composition for oral administration comprises purified water, which is removed during processing.

In some embodiments, the tablet comprises a coating between about 5% to about 20% (e.g., about 5%, 10%, 15% or 20%) by weight of the total weight of the tablet.

In the embodiment, the tablet comprises pregelatinized starch between about 4% to about 6% by weight of the total weight of the tablet.

In the embodiment, the tablet comprises colloidal silicon dioxide between about 1% to about 2.5% by weight of the total weight of the tablet.

In the embodiment, the tablet comprises magnesium stearate between about 0.5% to about 1.5% by weight of the total weight of the tablet.

In some embodiments, the tablet comprises pregelatinized starch, colloidal silicon dioxide, and magnesium stearate at a weight ratio of 5:2:1.

In some embodiments, the tablet comprises a coating of around 10% by weight of the tablet.

In some embodiments, the composition comprising Compound 1, or a pharmaceutically acceptable salt, solvate, or polymorph thereof is stored at controlled room temperature (20-25° C.).

In some embodiments, the composition comprising Compound 1, or a pharmaceutically acceptable salt, solvate, or polymorph thereof is protected from light.

In some other embodiments, the composition comprising Compound 1, or a pharmaceutically acceptable salt, solvate, or polymorph thereof is suitable for pediatric uses and can be taken by pediatric thalassemia patients.

In some embodiments, the composition comprising Compound 1, or a pharmaceutically acceptable salt, solvate, or polymorph thereof is taken with food.

### Dosing

Typical oral dosages range from about 0.001 to about 100 mg/kg body weight per day. Typical oral dosages also range from about 0.01 to about 50 mg/kg body weight per day. Typical oral dosages further range from about 0.05 to about 10 mg/kg body weight per day. Oral dosages are usually administered in one or more dosages, typically, one to three dosages per day. The exact dosage will depend upon the frequency and mode of administration, the gender, age, weight and general health of the subject treated, the nature and severity of the condition treated and any concomitant diseases to be treated and other factors evident to those skilled in the art.

In some embodiments, Compound 1, or a pharmaceutically acceptable salt, solvate, or polymorph thereof is administered to a patient in need thereof at a dosing of less than 6.0 mg/kg or less than about 4.0 mg/kg. For example, Compound 1, or a pharmaceutically acceptable salt, solvate, or polymorph thereof is administered at a dosing of between about 0.3 to about 3.0 mg/kg, or about 0.3 to about 1.0 mg/kg. The patient may have thalassemia, such as beta thalassemia. The patient may be an adult (>18 years old) or a child (<18 years old). In some embodiments, the patient receives Compound 1, or a pharmaceutically acceptable salt, solvate, or polymorph thereof at a dose of around 0.3 mg/kg, 0.2 mg/kg, 0.1 mg/kg, or 0.05 mg/kg.

In some embodiments, the patient receives Compound 1, or a pharmaceutically acceptable salt, solvate, or polymorph thereof at about 1 mg/kg. In some embodiments, the patient receives Compound 1, or a pharmaceutically acceptable salt, solvate, or polymorph thereof at about 3 mg/kg. In some embodiments, the patient receives Compound 1, or a pharmaceutically acceptable salt, solvate, or polymorph thereof at about 6 mg/kg.

In some embodiments, the patient receives Compound 1 or a pharmaceutically acceptable salt, solvate, or polymorph thereof at about 0.1 mg/kg.

In some embodiments, the patient receives Compound 1, or a pharmaceutically acceptable salt, solvate, or polymorph thereof at about 0.3 mg/kg.

In some embodiments, the patient receives Compound 1, or a pharmaceutically acceptable salt, solvate, or polymorph thereof at about 0.5 mg/kg.

In some embodiments, the patient receives Compound 1, or a pharmaceutically acceptable salt, solvate, or polymorph thereof at about 1.0 mg/kg.

In some embodiments, the patient receives Compound 1, or a pharmaceutically acceptable salt, solvate, or polymorph thereof at about 5.0 mg/kg.

In some embodiments, the patient receives Compound 1, or a pharmaceutically acceptable salt, solvate, or polymorph thereof at about 8.0 mg/kg.

In some embodiments, the patient receives Compound 1, or a pharmaceutically acceptable salt, solvate, or polymorph thereof at about 10 mg/kg.

In some embodiments, the patient receives Compound 1, or a pharmaceutically acceptable salt, solvate, or polymorph thereof at about 20 mg/kg.

In some embodiments, Compound 1, or a pharmaceutically acceptable salt, solvate, or polymorph thereof is administered to a patient in need thereof at a flat dose of about 20 mg, about 50 mg, about 100 mg, about 200 mg, about 300 mg, about 400 mg, about 500 mg, or about 600 mg per day.

In some embodiments, Compound 1 or a pharmaceutically acceptable salt, solvate, or polymorph thereof is administered to a patient, wherein Compound 1 is administered once a day.

In some embodiments, Compound 1, or a pharmaceutically acceptable salt, solvate, or polymorph thereof is administered to a patient, wherein Compound 1, or a pharmaceutically acceptable salt, solvate, or polymorph thereof is administered once a day with food. It has been found that food reduce the adverse event profile dramatically. The incidence and severity of the side effects, such as nausea, emesis and headache, can be reduced when Compound 1, or a pharmaceutically acceptable salt, solvate, or polymorph thereof is taken with food.

In some embodiments, Compound 1, or a pharmaceutically acceptable salt, solvate, or polymorph thereof is administered to a patient, wherein Compound 1, or a pharmaceutically acceptable salt, solvate, or polymorph thereof is administered once a day for at least 7 days, 10 days, 2 weeks, 3 weeks, 4 weeks, 1 month, 2 months, 3 months, 4 months, 6 months, 7 months, 8 months, 9 months, 10 months, 11 months, a year, 1.5 years, or 2 years. In some embodiments, the patients are treated for 3 months. In some embodiments, the patients are treated for 6 months. In some embodiments, the patients are treated for 1 year. In some embodiments, the patients are treated for 1.5 years. In some embodiments, the patients are treated for 2 years, 3 years, 4 years, 5 years, over 5 years, or the duration of life.

The formulations may also be presented in a unit dosage form by methods known to those skilled in the art. For illustrative purposes, a typical unit dosage form for oral administration may contain from about 0.01 to about 1000 mg, from about 0.05 to about 500 mg, or from about 0.5 mg to about 200 mg.

### III. Methods of Using Compounds of the Disclosure

One aspect of the present disclosure provides methods of using a PDE9 inhibitor, such as Compound 1, or a pharmaceutically acceptable salt, solvate, or polymorph thereof, to treat thalassemia. Thalassemias are genetic blood disorders. There are two main types, alpha thalassemia and beta thalassemia. Of these, there are multiple subtypes. Thalassemias are characterized by decreased hemoglobin (Hb) production. Symptoms depend on the type and can vary from no symptoms to sever symptoms. Often there is mild to severe anemia (low red blood cells or hemoglobin). Anemia can result in feeling tired, dizzy, weak, and pale or yellow skin. There may also be iron overload, increased rate of infections, bone deformities, an enlarged spleen, and heart problems such as congestive heart failure or abnormal heart rhythms. Slow growth and a delay in puberty may occur in children.

In some embodiments, the thalassemia is beta thalassemia. In some embodiments, the thalassemia is beta-plus thalassemia or beta-zero thalassemia. In some embodiments, the thalassemia is major beta thalassemia or minor beta thalassemia. In some embodiments, the thalassemia is alpha thalassemia. In some embodiments, the thalassemia is major alpha thalassemia or minor alpha thalassemia.

In another embodiment, Compound 1 is used to increase hemoglobin (Hb) levels in a subject. The Hb level may be increased by at least 5%, 10%, 25%, 50%, 100%, 150%, 200%, or 250%. In some embodiments, Compound 1 is used to increase Hb levels by about 2 times, 3 times, 4 times, 5 times, 10 times, 15 times, 20 times, or 25 times over Hb levels prior to treatment.

In some embodiments, the hemoglobin (Hb) levels of the subject are increased in the range of about 0.5 to about 3.0 g/dL of total Hb. In some embodiments, the hemoglobin (Hb) level of the subject is increased by about 0.5, about 1.0, about 1.5, about 2.0, about 2.5, or about 3.0 g/dL of total Hb.

In another embodiment, Compound 1 is used to increase fetal hemoglobin (HbF) levels in a subject. The HbF level may be increased by at least 5%, 10%, 25%, 50%, 100%, 150%, 200%, or 250%. In some embodiments, Compound 1 is used to increase HbF levels by about 2 times, 3 times, 4 times, 5 times, 10 times, 15 times, 20 times, or 25 times over baseline levels prior to treatment.

In another embodiment, Compound 1 is used to increase red blood cell (RBC) levels in a subject. The RBC level may be increased by at least 5%, 10%, 25%, 50%, 100%, 150%, 200%, or 250%. In some embodiments, Compound 1 is used to increase RBC levels by about 2 times, 3 times, 4 times, 5 times, 10 times, 15 times, 20 times, or 25 times over baseline levels prior to treatment.

In yet another embodiment, Compound 1 is used to increase mature RBC levels, reduce immature RBC levels, and/or increase maturation ratio. RBC maturation is measured by calculating the ratio of immature red blood cells (RBC) (Ery.B: late basophilic and polychromatic) in relation to mature RBC (Ery.C: ortochromatic and reticulocytes) i.e. as Ery.B/Ery.C. The mature RBC level may be increased by at least 5%, 10%, 25%, 50%, 100%, 150%, 200%, or 250%. In some embodiments, mature RBC level is increased by about 2 times, 3 times, 4 times, 5 times, 10 times, 15 times, 20 times, or 25 times over the baseline level prior to treatment. The immature RBC level may be reduced by at least 5%, 10%, 20%, 30%, 40%, 50%, 60%, 70%, or 80%. The maturation ratio may be increased by at least 5%, 15%, 25%, 50%, 100%, 150%, 200%, or 250%. In some embodiments, the maturation ratio is increase by about 2 times, 3 times, 4 times, 5 times, 10 times, 15 times, 20 times, or 25 times over the baseline ratio prior to treatment.

In some embodiments, the method increases hemoglobin levels, red blood cell levels, or levels of mature red blood cells greater than traditional blood transfusion.

### Combination Therapies

Another aspect of the present disclosure provides methods of using the PDE9 inhibitor of the present disclosure, such as Compound 1, or a pharmaceutically acceptable salt, solvate, or polymorph thereof, in combination with at least one other active agent. They may be administered simultaneously or sequentially. They may be present as a mixture for simultaneous administration, or may each be present in separate containers for sequential administration. One non-limiting example of one other active agent is folic acid (vitamin B) supplements.

The PDE9 inhibitor of the present disclosure, such as Compound 1, may also be used in combination with at least another therapy for thalassemia, such as blood transfusion, iron chelation, gene therapy, or blood and/or marrow stem cell transplant.

The term “simultaneous administration”, as used herein, is not specifically restricted and means that the PDE9 inhibitor of the present disclosure and the at least one other active agent are substantially administered at the same time, e.g. as a mixture or in immediate subsequent sequence.

The term “sequential administration”, as used herein, is not specifically restricted and means that the PDE9 inhibitor of the present disclosure and the at least one other active agent are not administered at the same time but one after the other, or in groups, with a specific time interval between administrations. The time interval may be the same or different between the respective administrations of PDE9 inhibitor of the present disclosure and the at least one other active agent and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

The molar ratio of the PDE9 inhibitor of the present disclosure and the at least one other active agent is not particularly restricted. For example, when a PDE9 inhibitor of the present disclosure and one other active agent are combined in a composition, the molar ratio of them may be in the range of 1:500 to 500:1, or of 1:100 to 100:1, or of 1:50 to 50:1, or of 1:20 to 20:1, or of 1:5 to 5:1, or 1:1. Similar molar ratios apply when a PDE9 inhibitor of the present disclosure and two or more other active agents are combined in a composition. The PDE9 inhibitor of the present disclosure may comprise a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the composition.

The other active agent may be a different PDE9 inhibitor of the present disclosure. The other active agent may also be an antibiotic agent such as penicillin, a nonsteroidal anti-inflammatory drug (NSAIDS) such as diclofenac or naproxen, a pain relief medication such as opioid, or folic acid. In some embodiments, the other active agent is folic acid.

Yet another aspect of the present disclosure provides methods of using a PDE9 inhibitor of the present disclosure in combination with at least one other therapy, such as but not limited to blood transfusion, bone marrow transplant, or gene therapy. In some embodiments, the additional therapy is a gene therapy. In some embodiments the additional therapy is a bone and/or marrow stem cell transplant. In some embodiments, the additional therapy is a blood transfusion. In some embodiments, the additional therapy is an iron chelation therapy or excess iron removal. Non limiting examples of iron chelating agents are deferoxamine (DFOA), Desferal, defersirox, Exjade, Desirox, Defrijet, Desifer, Rasiroxpine, and Jadenu. In some embodiments, the iron chelating agent is deferoxamine (DFOA). In some embodiments, the iron chelating agent is defersirox.

### IV. Kits and Devices

The disclosure provides a variety of kits and devices for conveniently and/or effectively carrying out methods of the present disclosure. Typically, kits will comprise sufficient amounts and/or numbers of components to allow a user to perform multiple treatments of a subject(s) and/or to perform multiple experiments.

In one embodiment, the present disclosure provides kits for treating thalassemia, comprising a PDE9 inhibitor compound of the present disclosure or a combination of PDE9 inhibitor compounds of the present disclosure, optionally in combination with any other active agents, such as folic acid, an antibiotic agent such as penicillin, a nonsteroidal anti-inflammatory drug (NSAIDS) such as diclofenac or naproxen, a pain relief medication such as opioid, or folic acid.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of PDE9 inhibitor compounds in the buffer solution over a period of time and/or under a variety of conditions.

The present disclosure provides for devices that may incorporate PDE9 inhibitor compounds of the present disclosure. These devices contain in a stable formulation available to be immediately delivered to a subject in need thereof, such as a human patient with thalassemia.

Non-limiting examples of the devices include a pump, a catheter, a needle, a transdermal patch, a pressurized olfactory delivery device, iontophoresis devices, multi-layered microfluidic devices. The devices may be employed to deliver PDE9 inhibitor compounds of the present disclosure according to single, multi- or split-dosing regiments. The devices may be employed to deliver PDE9 inhibitor compounds of the present disclosure across biological tissue, intradermal, subcutaneously, or intramuscularly. More examples of devices suitable for delivering PDE9 inhibitor compounds include but not limited to a medical device for intravesical drug delivery disclosed in International Publication WO 2014036555, a glass bottle made of type I glass disclosed in US Publication No. 20080108697, a drug-eluting device comprising a film made of a degradable polymer and an active agent as disclosed in US Publication No. 20140308336, an infusion device having an injection micropump, or a container containing a pharmaceutically stable preparation of an active agent as disclosed in U.S. Pat. No. 5,716,988, an implantable device comprising a reservoir and a channeled member in fluid communication with the reservoir as disclosed in International Publication WO 2015023557, a hollow-fibre-based biocompatible drug delivery device with one or more layers as disclosed in US Publication No. 20090220612, an implantable device for drug delivery including an elongated, flexible device having a housing defining a reservoir that contains a drug in solid or semi-solid form as disclosed in International Publication WO 2013170069, a bioresorbable implant device disclosed in U.S. Pat. No. 7,326,421, contents of each of which are incorporated herein by reference in their entirety.

### V. Definitions

The articles “a” and “an,” as used herein, should be understood to mean “at least one,” unless clearly indicated to the contrary.

The phrase “and/or,” as used herein, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements).

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements.

In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of” when used in the claims, shall have its ordinary meaning as used in the field of patent law.

As used herein, the phrase “at least one” in reference to a list of one or more elements should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, all transitional phrases such as “comprising,” “including,” “carrying,” “having,” “containing,” “involving,” “holding,” and the like are to be understood to be open-ended, i.e., to mean including but not limited to.

Only the transitional phrases “consisting of” and “consisting essentially of” shall be closed or semi-closed transitional phrases, respectively, as set forth in the United States Patent Office Manual of Patent Examining Procedures.

As used herein, a “subject” or a “patient” refers to any mammal (e.g., a human), such as a mammal that may be susceptible to a disease or disorder, for example, tumorigenesis or cancer. Examples include a human, a non-human primate, a cow, a horse, a pig, a sheep, a goat, a dog, a cat, or a rodent such as a mouse, a rat, a hamster, or a guinea pig. In various embodiments, a subject refers to one that has been or will be the object of treatment, observation, or experiment. For example, a subject can be a subject diagnosed with cancer or otherwise known to have cancer or one selected for treatment, observation, or experiment on the basis of a known cancer in the subject.

As used herein, “treatment” or “treating” refers to amelioration of a disease or disorder, or at least one sign or symptom thereof “Treatment” or “treating” can refer to reducing the progression of a disease or disorder, as determined by, e.g., stabilization of at least one sign or symptom or a reduction in the rate of progression as determined by a reduction in the rate of progression of at least one sign or symptom. In another embodiment, “treatment” or “treating” refers to delaying the onset of a disease or disorder.

As used herein, “prevention” or “preventing” refers to a reduction of the risk of acquiring or having a sign or symptom a given disease or disorder, i.e., prophylactic treatment.

The phrase “therapeutically effective amount” as used herein means that amount of a compound, material, or composition comprising a compound of the present teachings that is effective for producing a desired therapeutic effect. Accordingly, a therapeutically effective amount treats or prevents a disease or a disorder, e.g., ameliorates at least one sign or symptom of the disorder. In various embodiments, the disease or disorder is a cancer.

A dash (“-”) that is not between two letters or symbols is used to indicate a point of attachment for a substituent. For example, —CONH₂ is attached through the carbon atom (C).

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

All numerical ranges herein include all numerical values and ranges of all numerical values within the recited range of numerical values. As a non-limiting example, (C₁-C₆) alkyls also include any one of C₁, C₂, C₃, C₄, C₅, C₆, (C₁-C₂), (C₁-C₃), (C₁-C₄), (C₁-C₅), (C₂-C₃), (C₂-C₄), (C₂-C₅), (C₂-C₆), (C₃-C₄), (C₃-C₅), (C₃-C₆), (C₄-C₅), (C₄-C₆), and (C₅-C₆) alkyls.

Further, while the numerical ranges and parameters setting forth the broad scope of the disclosure are approximations as discussed above, the numerical values set forth in the Examples section are reported as precisely as possible. It should be understood, however, that such numerical values inherently contain certain errors resulting from the measurement equipment and/or measurement technique.

### List of Abbreviations and Terms

¹H-NMR: Proton Nuclear Magnetic Resonance spectroscopy

### ADME: Absorption, Distribution, Metabolism, and Excretion

AE: adverse event
AUC₀₋₂₄: area under the concentration-time curve from time 0 to 24 hours postdose
BBB: blood-brain barrier
Cₘₐₓ: maximum plasma concentration
cGMP: cyclic guanosine monophosphate
CNS: central nervous system
CHM: Cochran-Mantel-Haenzel test
CV: coefficient of variation
CYP: cytochrome p450

### DMC: Data Monitoring Committee

DMSO: dimethyl sulfoxide
DOAC: direct-acting oral anti-coagulant
ECG: electrocardiogram
EOT: end of treatment
FIH: first in human
FTIR: Fourier transform infrared spectroscopy
GC: gas chromatographs
Hb: hemoglobin
HBB: hemoglobin subunit beta
HBG: gamma-globin gene
HbE: hemoglobin E
HbS: hemoglobin S
hERG: human ether-à-go-go related gene
HPLC: high-performance liquid chromatographs
HU: hydroxyurea
IC: inhibitors- concentration
IC₅₀ : a half minimal inhibitors concentration
ICAM-1: intercellular adhesion molecule-1

### ICH: International Conference on Harmonization

### ICT: Iron Chelation Therapy

ICP-MS: inductively coupled plasma mass spectroscopy
IV: intravenous
MAD: multiple-ascending dose
MTD: maximum tolerated dose
NO: nitric oxide
NOAEL: no-observed-adverse-effect level
NTDT: Non-transfusion dependent beta thalassemia
NTDT-PRO: Non-transfusion dependent beta thalassemia patient report outcome
PD: pharmacodynamic
PDE9: phosphodiester-9
PEG polyethylene glycol

### P-gp: P-glycoprotein

PIC: Powder in capsule
PK: pharmacokinetic(s)
PKG: protein kinase G
pRBC: packed red blood cells
RBC: red blood cell
RH: relative humidity
qd: once daily
QoL: quality of life
SAD: single ascending dose
SAE: serious adverse event
SCD: sickle cell disease
SD: standard deviation
SEM: standard error of the mean
sGC: soluble guanylyl cyclase
t½: half-life
TDT: transfusion-dependent beta thalassemia

### TK: Toxicokinetic

Tₘₐₓ: time of maximum concentration
TranQoL: transfusion-dependent quality of life
ULN: upper limit of normal
VOC: vaso-occlusive crisis
WBC: white blood cell
w/w%: weight/weight percent

### EXAMPLES

It will be appreciated that the following examples are intended to illustrate but not to limit the present disclosure. Various other examples and modifications of the foregoing description and examples will be apparent to a person skilled in the art after reading the disclosure without departing from the spirit and scope of the disclosure, and it is intended that all such examples or modifications be included within the scope of the appended claims. All publications and patents referenced herein are hereby incorporated by reference in their entirety.

### Example 1. Synthesis and Formulation of Compound 1

Compound 1 is an enantiomer of 6-[4-methyl-1-(pyrimidin-2-ylmethyl)pyrrolidin-3-yl]-3-tetrahydropyran-4-yl-7H-imidazo[1,5-a]pyrazin-8-one disclosed in WO 2013/053690. Compound 1 may be prepared from chiral-selective purification from 6-[4-methyl-1-(pyrimidin-2-ylmethyl)pyrrolidin-3-yl]-3-tetrahydropyran-4-yl-7H-imidazo[1,5-a]pyrazin-8-one prepared according to the method disclosed in WO 2013/053690, the contents of which are incorporated herein by reference in their entirety. Compound 1 may also be prepared with the method disclosed in WO 2017/005786, the contents of which are incorporated herein by reference in their entirety.

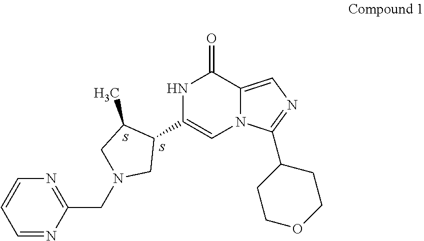

### Example 2. In Vivo Studies with Compound 1 for Treatment of Thalassemia

Hbb^{th1} mice do not have functional Hbβ leading to RBC/Hb deficits and defective RBC maturation. This model was used to evaluate the effect of Compound 1 to treat thalassemia.

Hbb^{th1} mice were given Compound 1 dissolved in 10:90 DMSO:corn oil once daily, by oral gavage, at either a high dose (60 mg/kg) or a low dose (30 mg/kg). A normal (C57BL/6) mouse was included as a control in the study to establish baselines along with a Hbb^{th1} vehicle mouse not given Compound 1. Animals were observed once daily for morbidity and mortality. Mice were not allowed to recover from anesthesia and were killed by cervical dislocation while still anesthetized. Necropsy was limited to removal and weighing of the spleen for further analysis.

Blood was collected on day 30, from the retroorbital sinus of all mice, while mice were under isoflurane anesthesia, using heparin capillaries and EDTA-coated tubes. Parameters measured were: Hb levels and RBC levels.

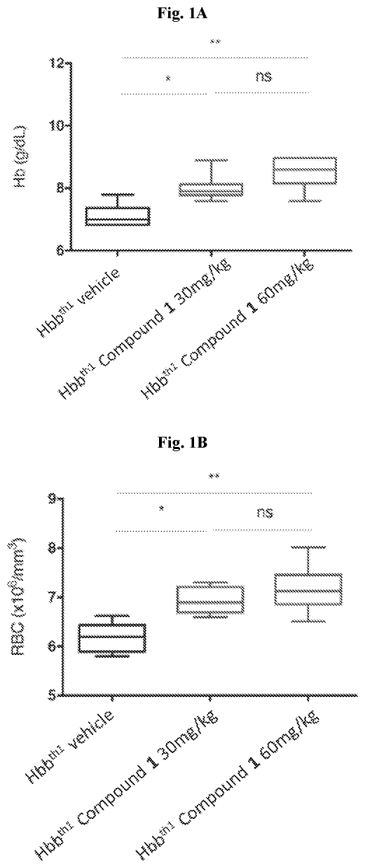

Comparative Hb levels are shown in FIG. 1A and RBC levels are shown in FIG. 1B. Compound 1 treatments increase Hb levels. Compound 1 high dose shows an increase of 1.3 g/dL of total Hb vs. control groups, which is better than traditional transfusion (an increase of around 1 g/dL of total Hb). Compound 1 treatments also increased RBC levels (FIG. 1B).

RBC maturation is a key mechanistic component in reducing pathology of thalassemia. Therefore, immature RBC (Ery.B: late basophilic and polychromatic) percentage, mature RBC (Ery.C: ortochromatic and reticulocytes) percentage, and maturation index were also measured. To measure erythroblast differentiation, single-cell suspensions from Hbb^{th1} mice spleen were prepared without enzymatic treatment using a GentleMacs dissociator (Miltenyi Biotec). The cell suspension was filtered through a 30 μm cell strainer and incubated with mouse Fc Blocking reagent (Miltenyi Biotec) to block IgG receptors. Cells (1×10⁶) were then stained with antibodies against TER-119 (Biolegend clone Ter-119 APC-Cy7 #116223) and mouse CD71 (Biolegend clone RI7217 FITC #113806). Dead cells were excluded by Live-Dead Aqua labeling (Life Technologies). Cells were further analyzed by flow cytometry (Gallios, Beckman) using FlowJo software v.10.1 (FlowJo, LLC). Results were expressed as the percentage of Ery.B (Live-Dead^{neg}, Ter-119^{pos}, CD71^{pos}, FSC^{low}) or Ery.C (Live-Dead^{neg}, Ter-119^{pos}, CD71^{neg}, FSC^{low}) populations. Maturation index was calculated as Ery.B/Ery.C ratio.

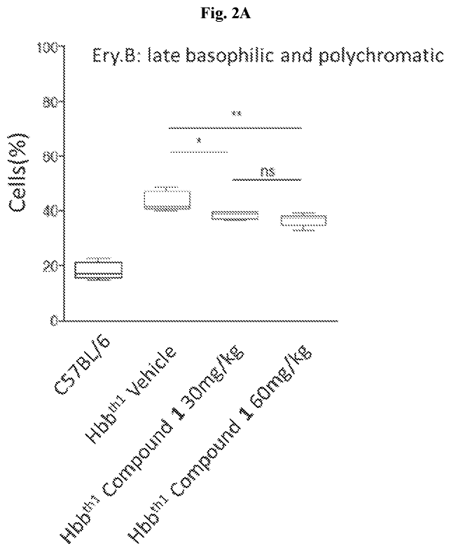

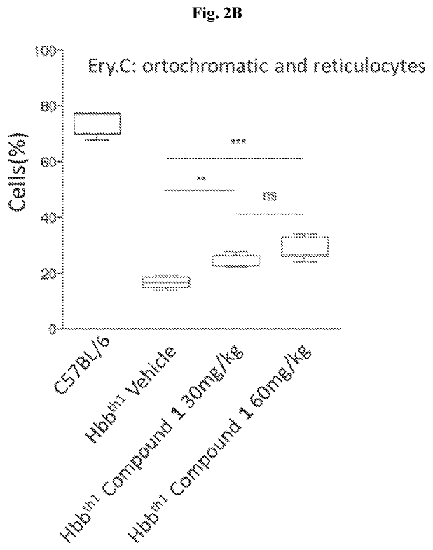

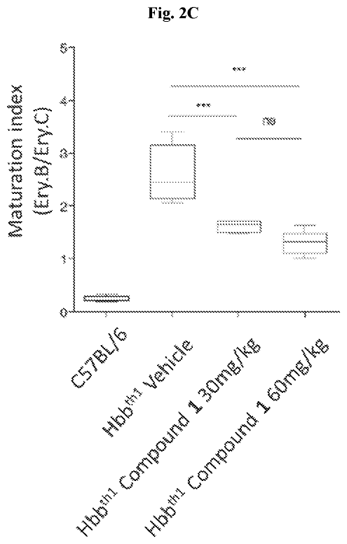

As shown in FIG. 2A, FIG. 2B and FIG. 2C, Compound 1 treatments reduces immature RBC cell percentages, increases mature RBC cell percentages, and reduces maturation ratios.

### Example 3: Clinical Trials for Beta Thalassemia

A Phase 2 Study to evaluate the safety and tolerability of Compound 1 in subjects with beta thalassemia will be conducted. This study will be conducted study sites in North America, United Kingdom, European Union, Middle East, Asia-Pacific, and Africa.

Primary Objective: The primary objective of the study in both Population 1, transfusion-dependent beta thalassemia (TDT) and Population 2, non-transfusion-dependent beta thalassemia (NTDT) is to assess the safety and tolerability of Compound 1 in adult subjects with beta thalassemia.

Secondary Objectives: The secondary objectives in Population 1 (TDT) subjects are: 1) To evaluate the effect of Compound 1 versus placebo on the reduction in red blood cell (RBC) transfusion burden. 2) To evaluate the change in transfusional iron load rate of Compound 1 versus placebo. 3) To characterize the pharmacokinetic (PK) profile of Compound 1.

The secondary objectives in Population 2 (NTDT) subjects are: 1) Evaluate the effect of Compound 1 versus placebo on fetal hemoglobin (HbF). 2) To evaluate the effect of Compound 1 versus placebo on anemia. 3) To characterize the PK profile of Compound 1.

Exploratory Objectives: The exploratory objectives in Population 1 (TDT) subjects are: 1) To characterize the pharmacodynamic (PD) profile of Compound 1 versus placebo. 2) To evaluate the effect of Compound 1 versus placebo on the mean change from baseline in transfusion burden. 3) To evaluate the effect of Compound 1 versus placebo on the proportion of subjects who are transfusion independent. 4) To evaluate the effect of Compound 1 versus placebo on time and duration of transfusion burden. 5) To evaluate the effect of Compound 1 versus placebo on the characteristics of the erythroid response. 6) To evaluate the effect of Compound 1 versus placebo on functional and health-related quality of life (QoL). 7) To evaluate responses to Compound 1 per genotypes in subjects with TDT.

The exploratory objectives in Population 2 (NTDT) subjects are: 1) To characterize the PD profile of Compound 1 versus placebo. 2) To evaluate the effect of Compound 1 versus placebo on time and duration of transfusion burden. 3) To evaluate the effect of Compound 1 versus placebo on functional and health-related QoL. 4) To evaluate the effect of Compound 1 versus placebo on beta-thalassemia-related symptoms and severity. 5) To evaluate responses to Compound 1 per genotypes in subjects with NTDT.

Study Design (Methodology): This is a phase 2, randomized, double-blind, placebo-controlled study to evaluate the safety, tolerability, PK, and PD of Compound 1 administered once daily (qd) for 36 weeks in 2 populations of adult subjects with beta-thalassemia: Population 1 (TDT subjects) and Population 2 (NTDT subjects).

This study will enroll approximately 120 subjects with beta-thalassemia (60 subjects with TDT and 60 subjects with NTDT), aged 18 through 65 years. This study consists of a retrospective data collection period, a screening period, a double-blind treatment period, and a safety follow-up period. During the screening period of up to 28 days, subjects will provide informed consent and be evaluated on eligibility criteria as stated below.

Subjects will receive either Compound 1 (lower dose or higher dose) or placebo in a blinded fashion. Subjects will be randomly assigned in a 2:1 ratio to receive either Compound 1 lower dose or placebo.

Subjects will return to the investigational site at Week 1 for a safety assessment, and qualified site personnel will contact the subject by telephone at Week 2 to capture potential AEs and concomitant medications. Subjects will be seen at the investigational site approximately every 3 weeks (TDT subjects) or every 4 weeks (NTDT subjects) throughout the remainder of the study. Safety will be monitored throughout the study, and PK, PD, QoL, and clinical outcome measures will be performed at the visits shown in the schedule of assessments for the TDT and NTDT populations.

The informed consent will specifically indicate that data will be retrospectively collected on transfusion burden, defined as the date of transfusion episodes and number of packed RBC units as issued at each transfusion during the 12 weeks preceding the Screening visit. If available, the following data will be collected for each transfusion episode: mean volume of pRBC units as issued (±SD or range in mL), pre-transfusion Hb (e.g., assessed at the time of crossmatch) as well as hematocrit from the blood bank for comparison, and subject's pre-transfusion weight.

Study Rationale: This is a phase 2 study intended to explore the potential use of Compound 1 to treat subjects with beta-thalassemia. This is the first study of Compound 1 in a beta-thalassemia population, and, as such, is designed to examine the safety, tolerability, and PK, as well as the potential PD effects, of Compound 1 administered qd for 36 weeks in adult subjects with beta-thalassemia.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating thalassemia comprising administering 6-[4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or polymorph thereof to a subject in need thereof.

2. The method of claim 1, wherein the administration results in an increase of the hemoglobin level (Hb) of the subject.

3. The method of claim 2, wherein the subject's hemoglobin level (Hb) is increased in the range of about 0.5 to about 3.0 g/dL of total Hb.

4. The method of claim 3, wherein the subject's hemoglobin level (Hb) is increased by about 0.5, about 1.0, about 1.5, about 2.0, about 2.5, or about 3.0 g/dL of total Hb.

5. The method of any one of claims 1 to 4, wherein the administration results in an increase of the red blood cell (RBC) level of the subject.

6. The method of claim 5, wherein the RBC level is increased by at least 5%, 10%, 25%, or 50% over baseline RBC level prior to the administration of Compound 1.

7. The method of any one of claims 1 to 6, wherein the administration results in a decrease of the immature red blood cell level of the subject.

8. The method of claim 7, wherein the immature red blood cell level of the subject is decreased by at least 5%, 10%, 25%, or 50% over baseline level prior to the administration of Compound 1.

9. The method of any one of claims 1 to 8, wherein the administration results in an increase of the mature red blood cell level of the subject.

10. The method of claim 9, wherein the mature red blood cell level of the subject is increased by at least 5%, 10%, 25%, or 50% over baseline level prior to the administration of Compound 1.

11. The method of any one of claims 1 to 10, wherein the administration results in an increase in the red blood cell (RBC) maturation ratio of the subject prior to administration.

12. The method of claim 11, wherein the red blood cell (RBC) maturation ratio is increased by at least 5%, 10%, 25%, or 50%.

13. The method of any one of claims 1 to 12, wherein the method increases hemoglobin (Hb) levels, red blood cell (RBC) levels, or levels of mature red blood cells greater than traditional blood transfusion.

14. The method of any one of claims 1 to 13, wherein Compound 1, or a pharmaceutically acceptable salt, solvate, or polymorph thereof, is administered orally.

15. The method of any one of claims 1 to 14, wherein Compound 1, or a pharmaceutically acceptable salt, solvate, or polymorph thereof, is administered daily.

16. The method of any one of claims 1 to 15, wherein Compound 1, or a pharmaceutically acceptable salt, solvate, or polymorph thereof, is administered with food or without food.

17. The method of any one of claims 1 to 16, wherein Compound 1, or a pharmaceutically acceptable salt, solvate, or polymorph thereof, is administered for between 1 to 7 days.

18. The method of any one of claims 1 to 17, wherein Compound 1, or a pharmaceutically acceptable salt, solvate, or polymorph thereof, is administered for at least 7 days.

19. The method of any one of claims 1 to 18, wherein Compound 1, or a pharmaceutically acceptable salt, solvate, or polymorph thereof, is administered with an additional therapy.

20. The method of claim 19, wherein the additional therapy is a gene therapy a bone and/or marrow stem cell transplant a blood transfusion, or an iron chelation therapy.

21. The method of any one of claims 1 to 20, wherein the thalassemia is beta thalassemia.

22. The method of any one of claims 1 to 21, wherein the thalassemia is beta-plus thalassemia or beta-zero thalassemia.

23. The method of any one of claims 1 to 22, wherein the thalassemia is major beta thalassemia or minor beta thalassemia.

24. The method of any one of claims 1 to 20, wherein the thalassemia is alpha thalassemia.

25. The method of any one of claim 1 to 20 or 24, wherein the thalassemia is major alpha thalassemia or minor alpha thalassemia.
